(19) United States
(12) Patent Application Publication
(10) Pub. No.: US 2012/0148493 A1
(43) Pub. Date: Jun. 14, 2012

(54) Composite Materials Loaded with Therapeutic and Diagnostic Agents Comprising Polymer Nanoparticles and Polymer Fibers

(75) Inventors: Thomas Schmehl, Giessen (DE); Juliane Nguyen, Wallenhorst (DE); Moritz Beck-Broichsitter, Marburg (DE); Tobias Gessler, Wettenberg (DE); Thomas Kissel, Staufen (DE); Marcel Thieme, Northeim (DE)

(73) Assignee: Justus-Liebig-Universitat Giessen, Giessen (DE)

(21) Appl. No.: 13/256,872

(22) PCT Filed: Mar. 16, 2010
(86) PCT No.: PCT/EP10/53381

§ 371 Date: Mar. 5, 2012

(30) Foreign Application Priority Data
Mar. 16, 2009 (DE) 102009013012.8

(51) Int. Cl. A61K 9/70; A61P 9/00; A61P 11/00; A61P 11/06; A61P 35/00; A61P 17/00; A61P 17/02; D01D 5/00; A61K 49/00; A61P 3/00; B82Y 5/00

(52) U.S. Cl. 424/9.1; 424/443; 264/465; 977/773; 977/906; 977/904; 977/762; 977/895; 977/888

(57) ABSTRACT

The invention relates to composite materials comprising polymer nanofibers and polymer nanoparticles, wherein at least one of the two polymer materials is loaded with a substance selected from therapeutic and diagnostic agents. Fibers and nanoparticles can comprise identical or different polymers; the polymer materials are, however, biocompatible in every case. Therapeutic and diagnostic agents can be hydrophilic or lipophilic and the two polymer materials likewise. The at least one polymer material and the substance with which said material is loaded are either both hydrophilic or both lipophilic. The polymer nanoparticles of the composite materials have a diameter of 10 nm to 600 nm. The polymer fibers have diameters of 10 nm to 50 μm and lengths of 1 μm to several meters. The invention further relates to a method for producing said composite materials. Polymer nanoparticles can be produced in different ways, such as through controlled precipitation of a polymer solution that optionally comprises a loading substance. The nanoparticles are then mixed with another polymer and a loading substance as applicable, depending on whether particles, fibers or both are to be loaded with substance. The processing of this solution into composites comprising polymer fibers polymer nanoparticles can occur by means of electrospinning, melt spinning, extruding or template process. Composite materials according to the invention are suitable for the production of pharmaceuticals that release therapeutically or diagnostically effective substances slowly and in a controlled manner.

The present invention relates to composite materials comprising polymer nanoparticles and polymer fibers, wherein at least one of the two polymer materials is loaded with a substance selected from therapeutic and diagnostic agents. Fibers and nanoparticles can comprise the same or different polymers. Therapeutic and diagnostic agents can be hydrophilic or lipophilic and the two polymer materials likewise. The at least one polymer material and the substance with which said material is loaded are either both hydrophilic or both lipophilic.

The present invention further relates to a method for producing said composite material.

Composite materials according to the invention are suitable for the production of pharmaceuticals that release therapeutically or diagnostically effective substances released slowly and in a controlled manner.

### BACKGROUND OF THE INVENTION

The present invention relates to the areas of polymer chemistry, pharmacy and medicine.

### PRIOR ART

Biocompatible polymer nanoparticles or polymer nanofibers are gaining increasing importance for the encapsulation of active pharmaceutical ingredients, because they enable controlled release applications, where the drug is not released by burst release, but in a controlled manner over a prolonged period of time. Active ingredients are high molecular weight or low molecular weight substances, which cause at low dose a specific reaction to occur in an organism.

The prior art knows, for example, methods for encapsulating active ingredients in very small nanoparticles. The disadvantage here is that active ingredient-containing nanoparticles with diameters below 200 nm release the active ingredient initially by burst release, as described in A Sheik Hasan, M Socha, A Lamprecht, F El-Ghazouani, A Sapin, M Hoffman, P Maincent and N Ubrich: “Effect of the microencapsulation on the reduction of burst release”, Int J Pharm 2007, 344, 53-61. According to Sheik Hasan et al., this may be prevented only if nanoparticles are encapsulated in microparticles, so that the resulting particles have a polymer double wall. Even drug-loaded fibers show a burst release, as described in K Kim, Y K Luu, C Chang, D Fang, B S Hsiao, B Chu and M Hadjiargyrou: “Incorporation and controlled release for a hydrophilic antibiotic using poly(lactide-co-glycolide)-based electrospun nanofibrous scaffolds”, J Control Release 2004, 98, 47-56.

Also, polymer fibers with very small diameters are described as a carrier for pharmaceuticals. In DE 10 2005 056 490 A1 micro- or nanofibers or hollow micro- or nanofibers are described that contain particles that are excitable in a magnetic field, wherein at least a portion of the fiber material is soluble in a liquid medium. These fibers are intended to serve as a component of a pharmaceutical in hyperthermia and/or thermal ablation.

In S Maretschek, A Greiner and T Kissel: “Electrospun biodegradable nanofiber nonwovens for controlled release of proteins”, J Control Release 2008, 127, 180-187, electrospun poly-L-lactide-/polyethyleneimine blends for encapsulating cytochrome c are described. These drug-containing polymer nonwovens are very hydrophobic and therefore are suitable only for the encapsulation of hydrophobic active ingredients.

DE 10 2006 061 539 A1 describes agents for delivering active ingredients to a wound or to the skin surrounding the wound. Said agents comprise at least a first layer of a fiber material and a wound-healing substance present in the form of particles. The particles may comprise a carrier material and the wound-healing substance, wherein the particles act as a depot for the controlled release. The particle diameter is between 1 μm and 1000 μm. Optionally, the fiber material may be a nonwoven made of staple fibers.

Thus far, the prior art does not know any agents for the encapsulation of therapeutic and diagnostic agents, wherein lipophilic and hydrophilic properties of biocompatible polymers can be combined.

The present invention overcomes these disadvantages by providing for the first time a biocompatible composite material comprising polymer fibers and polymer nanoparticles, wherein at least one of said two polymer materials is loaded with therapeutic or diagnostic agents.

### OBJECTIVE TECHNICAL PROBLEM

The objective technical problem of the invention is to provide new biocompatible agents for immobilization and for prevention of the burst release effect of therapeutic and diagnostic agents and a process for their production.

### SOLUTION OF THE PROBLEM

The task of providing biocompatible agents for immobilization and the prevention of the burst release effect of therapeutic and diagnostic agents is solved by composite materials according to the invention comprising polymer fibers and polymer nanoparticles, wherein
polymer nanoparticles and polymer fibers comprise biocompatible polymers,
at least one of the polymer materials is loaded with at least one substance selected from therapeutic and diagnostic agents
therapeutic and diagnostic agents are selected from hydrophilic and lipophilic substances
   characterized in that
the polymer nanoparticles have diameters of 10 nm to 600 nm,
the polymer fibers have diameters of 10 nm to 50 μm and lengths of 1 μm to several meters,
the polymer nanoparticles comprise a first polymer and the nanofibers comprise a second polymer,
first and second polymer is selected from hydrophilic and lipophilic polymers,
first and second polymer are identical or different and
the at least one polymer material and the at least one substance, with which it is loaded, are both hydrophilic, or are both lipophilic.

Surprisingly, it was found, that the above-described composite materials comprising polymer nanoparticles and polymer fibers, wherein at least one of these polymer materials is loaded with at least one substance selected from therapeutic and diagnostic agents, release these substances not in form of a burst release, but delayed. So far, known nanoparticles loaded with a therapeutic or diagnostic agent with diameters in the sub-micrometer range release the active ingredient in form of a burst release. In contrast, the composites according to the invention do not exhibit a burst release effect, but a controlled release effect.

Composite materials according to the invention and the process for producing them are explained below, with the invention comprising all embodiments listed below, individually and in combination.

The term “composite” generally refers to composite materials.

Accordingly, composite materials according to the invention comprise polymer fibers and polymer nanoparticles, wherein at least one of said polymer materials is loaded with at least one substance selected from therapeutic and diagnostic agents.

Hereinafter, nanoparticles are designated “NP”.

The term “therapeutic agent” in the context of the present invention is understood to mean high molecular weight or low molecular weight molecules that in certain dose regimens are used to cure, mitigate or prevent diseases. By contrast, diagnostic agents are high molecular weight or low molecular weight substances that serve the detection of a disease as a nosological entity.

Both therapeutic agents and diagnostic agents include on one hand substances whose primary effect as intended is achieved by pharmacologically or immunologically active agents and/or by metabolism, and on the other hand substances whose primary effect as intended is not achieved by said agents and/or by metabolism, their modes of action, however, can be supported by such agents. The present invention includes therapeutic and diagnostic agents with any of these two modes of action.

High molecular therapeutics include, for example, proteins and nucleic acids. Low molecular therapeutics include, for example, but are not limited to those selected from antibiotics, vitamins, cytostatic agents, virostatic agents, immunosuppressants, analgesics, anti-inflammatory drugs, proteolytics, vasoactive substances.

Magnetic particles are also included in the substances in the context of the present invention.

It is well known that such particles are used, for example in diagnostic imaging procedures, but also in therapy, for example in chemo- and radiotherapy, and in hyperthermia.

Diagnostic agents include in vitro and in vivo diagnostic agents. A diagnostic agent used according to the invention may be, for example, imaging and/or radioactive and/or a contrast agent.

Furthermore, both high molecular weight and low molecular weight therapeutic and diagnostic agents may be lipophilic or hydrophilic.

The person skilled in the art knows numerous therapeutic and diagnostic agents. The person skilled in the art can use them without departing from the scope of the claims.

According to invention, polymer nanoparticles comprise a first polymer and the polymer fibers comprise a second polymer, wherein the polymers are selected from hydrophilic and lipophilic polymers. Hereby, first and second polymer may be identical or different. Both the first and the second polymer are selected from biocompatible polymers.

Polymer nanoparticles and polymer fibers are collectively termed “polymer materials”.

In one embodiment, the first and second polymer are identical. In said embodiment, the polymer nanoparticles and the polymer fibers inevitably are either both hydrophilic or both lipophilic.

In another embodiment, the first and second polymer are different. In this embodiment, both polymers can be hydrophilic, both can be lipophilic or one can be hydrophilic and the other can be lipophilic.

In a preferred embodiment, the first and second polymer are different, one being hydrophilic and the other being lipophilic.

Optionally, at least one of the two polymers is not only biocompatible, but also biodegradable. Preferably, both polymers are biodegradable.

Biocompatible lipophilic polymers include silicones, poly (ethylene-co-vinyl acetate) and polyacrylates, resins (e.g. epoxyresins), silanes, siloxanes, nylon, polyethylene, polypropylene, polyamines, polyphosphazones, polybutene, polybutadienes, polyether, polyisoprenes.

Biocompatible lipophilic polymers, that are biodegradable, include, for example, polyesters, polyanhydrides, polyorthoesters, polyphosphoric acid ester, polycarbonates, polyketals, polyureas, polyurethanes.

Lipophilic polymers may include also block copolymers, PEG-PLGA, star polymers and/or comb polymers.

Hydrophilic polymers include, for example, polyethylene glycol, polyethylene imine, polyvinyl alcohol, polyvinyl acetate, polyvinyl butyral, polyvinyl pyrrolidone, polyacrylates and natural polymers such as proteins (e.g. albumin), celluloses and their esters and ethers, amylose, amylopectin, chitin, chitosan, collagen, gelatin, glycogen, polyamino acids (e.g. polylysine), starch, modified starches (e.g. HES), dextrans, heparins.

According to the invention, at least one of the polymer materials is loaded with at least of one substance selected from therapeutic and diagnostic agents. Hereby, the at least one polymer material and the at least one substance are both either hydrophilic or lipophilic.

In one embodiment, the polymer nanoparticles are loaded with at least one substance selected from therapeutic and diagnostic agents loaded, while the polymer fibers are not loaded.

In another embodiment, the polymer fibers are loaded with at least one substance selected from therapeutic and diagnostic agents, while the polymer nanoparticles are not loaded.

In another embodiment, both the polymer nanoparticles and the polymer nanofibers are loaded with at least one substance selected from therapeutic and diagnostic agents. Optionally, the polymer nanoparticles and the polymer fibers are loaded with different substances.

In a preferred embodiment, the polymer nanoparticles are loaded with exactly one substance selected from therapeutic and diagnostic agents, while the polymer fibers are not loaded.

In another preferred embodiment, both the polymer nanoparticles and the polymer fibers are loaded each with exactly one substance selected from therapeutic and diagnostic agents, wherein the fibers are loaded with a fast-releasing substance and the particles are loaded with a slow-releasing substance.

In another preferred embodiment, at least the polymer nanoparticles are loaded, and the first polymer and the at least one substance selected from therapeutic and diagnostic agents, with which the particles are loaded, are both lipophilic.

According to the invention, the second polymer that makes up the polymer fibers, may be cross-linked or not cross-linked.

In an embodiment, said second polymer is not cross-linked.

In another embodiment, the second polymer that makes up the fibers is cross-linked. This may be chemical or physical cross-linking.

A person skilled in the art knows how to crosslink polymers. The person skilled in the art may apply this knowledge without leaving the scope of the patent claims.

For example, alcohols such as polyvinyl alcohol can be cross-linked chemically with aldehydes or other cross-linkers.

Furthermore, polyvinyl alcohol may also be cross-linked physically by subjecting it to several hot-cold cycles. Another possibility for physical cross-linking involves irradiation with UV light.

Optionally, said polymer fibers may also be so-called nanowires, comprising an inner cylinder and a coating layer around it. Such nanowires are known in the art.

The polymer nanoparticles have diameters between 10 nm and 600 nm, preferably between 50 nm and 200 nm. In case of loaded polymer nanoparticles, the diameter depends on both the first polymer used and the therapeutic/diagnostic agent. In this case, of course, the specified lower limit of the particle diameter can only be reached with corresponding low molecular weight therapeutic and diagnostic agents, as can easily be calculated by a person skilled in the art using know molecular parameters of these substances.

The polymer fibers have diameters of 10 nm to 50 μm and lengths of 1 μm to several meters.

According to the invention, the objective of providing a process for producing the composite materials according to the invention is solved by a process comprising the following steps:
a) producing nanoparticles from a first polymer, wherein the nanoparticles are optionally loaded with at least one substance selected from therapeutic and diagnostic agents,
b) mixing of the optionally loaded polymer nanoparticles of step a) with a second polymer,
c) optionally adding at least one substance selected from therapeutic and diagnostic agents, wherein at least in one of steps a) and c) a substance selected from diagnostic and therapeutic agents is added,
d) processing the mixture of step c) into composites comprising polymer fibers and polymer nanoparticles.

Polymer nanoparticles can be produced, for example, by CVD, PVD, spray pyrolysis, sol-gel methods and controlled precipitation. When loaded nanoparticles are produced, the substance selected from therapeutic and diagnostic agents is added to the first polymer prior to the formation of nanoparticles. When producing the polymer nanoparticles according to the invention by spray pyrolysis, the first polymer and the substance used for loading must have sufficient thermal stability. The person skilled in the art knows suitable polymers, and therapeutic and diagnostic agents.

In a preferred embodiment of the present invention, the polymer nanoparticles are produced by controlled precipitation. First polymer and loading substance are mixed in a solvent with stirring and the resulting loaded polymer nanoparticles are then precipitated and separated. The polymer and the loading substance may initially be dissolved separately and the two solutions may then be mixed, or polymer and solvent may be dissolved together. In the case of producing two separate solutions, it is advantageous to use the same solvent for both solutions.

According to step b) of the process according to the invention, nanoparticles obtained from step a) are mixed with a second polymer. Optionally, a substance selected from therapeutic and diagnostic agents may be added to said mixture if loaded fibers are to be produced. In at least one of steps a) and c) a loading substance must be added, since in composites according to the invention at least one of the polymer materials is loaded with at least one substance selected from therapeutic and diagnostic agents.

The mixture of polymer nanoparticles, second polymer and optional loading substance according to step c) is subsequently processed to composites comprising polymer fibers and polymer nanoparticles. This may be carried out by, for example, electro-spinning, melt-spinning, extrusion or by template processes. The one skilled in the art knows that polymer nanofibers can be produced using said processes. If, nanoparticles are added to the polymer before processing into fibers, composites comprising polymer fibers and nanoparticles are obtained. If the composites according to the invention are produced by electro-spinning, extrusion or template processes, the mixture of polymer nanoparticles, second polymer and optional loading substance is produced according step c) in a solvent in which the second polymer is soluble.

However, where the polymer fibers are nanowires, they are suitably produced by co-electro-spinning, whereby a polymer forming the inner cylinder of the nanowires, and another polymer, forming the coating layer are spun together. This co-spinning is known to the person skilled in the art and can be used without leaving the scope of the patent claims.

The person skilled in the art knows that hydrophilic polymers or therapeutic and diagnostic agents are dissolved advantageously in hydrophilic solvents (same polarity) and are precipitated with lipophilic solvents (opposite polarity) and that it is the opposite in case of lipophilic polymers and therapeutic and diagnostic agents, respectively.

According to the invention, a polymer or a therapeutic and diagnostic agent is “soluble” in a solvent, if at least 0.1% by weight of it can be dissolved therein.

Accordingly, a polymer or therapeutic and diagnostic agent is “insoluble” in a solvent, if less than 0.1% by weight can be dissolved therein.

In a preferred embodiment, the polymer nanoparticles are produced by controlled precipitation and composites according to the invention are produced by electro-spinning.

When spinning hydrophilic polymer nanoparticles into hydrophilic polymer fibers (solid in water in an organic solvent) or lipophilic particles into lipophilic fibers (solids in an organic solvent in water), it is recommended to add biocompatible emulsifiers to the spinning solution. The biocompatible emulsifier can be, for example, a nonionic surfactant such as Tween or Span, an anionic surfactant such as a bile acid salt, an amphoteric surfactant such as lecithin, or a cationic surfactant.

The spinning solutions of the dissolved second polymer and the suspension of the nanoparticles can be electro-spun in any way known to a person skilled in the art, for example, by extrusion of the solution under low pressure through a cannula connected to a pole of a voltage source onto a counter electrode arranged at a distance to the cannula exit. Preferably, the distance between the cannula and the counter electrode which is acting as a collector and the voltage between the electrodes is set such that an electric field of preferably 0.5 to 2.5 kV/cm, more preferably 0.75 to 1.5 kV/cm and most preferably 0.8 to 1 kV/cm is formed between the electrodes.

Good results are obtained in particular when the inner diameter of the cannula is 50 to 500 μm.

The composite materials according to the invention can be used for the production of pharmaceuticals or medicinal products for patients for the treatment and prophylaxis of diseases, where a slow release (controlled release) of the pharmaceutically active ingredient is desirable.

This particularly applies to embodiments according to the invention, where polymer nanoparticles are loaded with therapeutic or diagnostic agents. Since the NP are spun into fibers, they are immobilized. The fibers prevent a burst release of the active ingredients by the nanoparticles.

Diseases include, for example, cardiovascular diseases, pulmonary diseases such as COPD, asthma, pulmonary hypertension. Furthermore included are disorders of the lipid metabolism, tumor diseases, congenital metabolic disorders (e.g. growth disorders, storage disorders, disorders of iron balance), endocrinological diseases, such as diseases of the pituitary gland or thyroid gland.

In addition, the composite materials according to the invention can be used to produce pharmaceuticals and medicinal products for the treatment of dermatological diseases, for wound healing, pain management, and as ophthalmological or contraceptive agents.

Furthermore, the composite materials according to the invention can be used to produce pharmaceuticals and medicinal products for the treatment of mental disorders (e.g. schizophrenia, depression, bipolar affective disorders, post-traumatic stress syndrome, anxiety and panic attacks) and for the treatment of CNS disorders by providing the composite materials, for example, as nonwovens for intracranial application.

In addition, the composite materials according to the invention can be used to produce pharmaceuticals and medicinal products for the treatment of diabetes, for example, in the form of depot insulin, or for the treatment of infectious diseases, for example by loading them with antibiotics. The composite materials according to the invention can also be used to produce pharmaceuticals and medicinal products for the treatment of allergic and autoimmune diseases (e.g. allergic asthma), and erectile dysfunction.

The term patient refers both to humans and vertebrates. Thus, the pharmaceuticals may be used both in human and veterinary medicine. Pharmaceutically acceptable compositions of composite materials according to the claims can be used, provided, after careful medical assessment, they cause no undue toxicity, irritation or allergic reactions in the patient. The therapeutically active compounds according to the present invention can be administered to the patient as part of a pharmaceutically acceptable composition either orally, buccally, sublingually, rectally, parenterally, intravenously, intramuscularly, subcutaneously, intracisternally, intravaginally, intraperitoneally, intravascularly, intrathecally, intravesically, topically, locally (powders, ointments or drops), or in spray form (aerosol). Intravenous, subcutaneous, intraperitoneal or intrathecal administration may be continuously using a pump or dosing unit. Dosage forms for local administration of compounds according to the invention include ointments, powders, suppositories, sprays, inhalants, patches, wound dressings, implants, and ophthalmic agents. Under sterile conditions the active component is mixed with a physiologically acceptable carrier and possible stabilizing and/or preserving additives, buffers, diluents, and propellants, as needed. For pulmonary (for example, in aerosol form as a spray) or peroral administration of the composite materials, the fiber mats obtained by the process according to the invention are first reduced to small pieces.

Furthermore, composite materials according to the invention can be used for tissue engineering.

### FIGURE KEY

FIG. 1

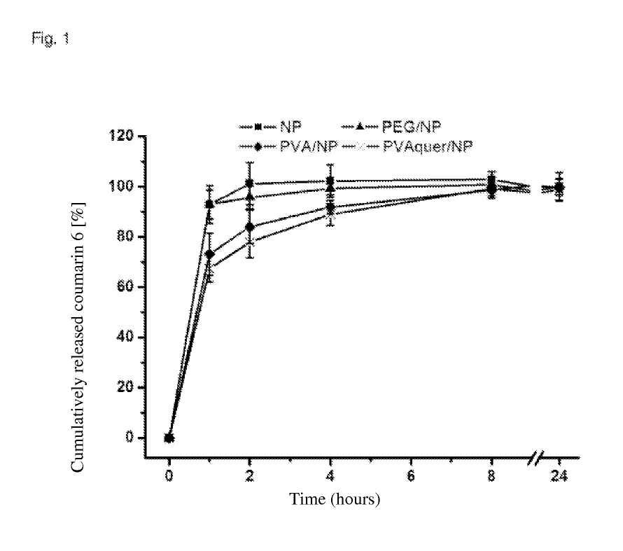

FIG. 1 shows the experiments for the release of coumarin 6 from particles and from particles spun into fibers (10% w/w), respectively.

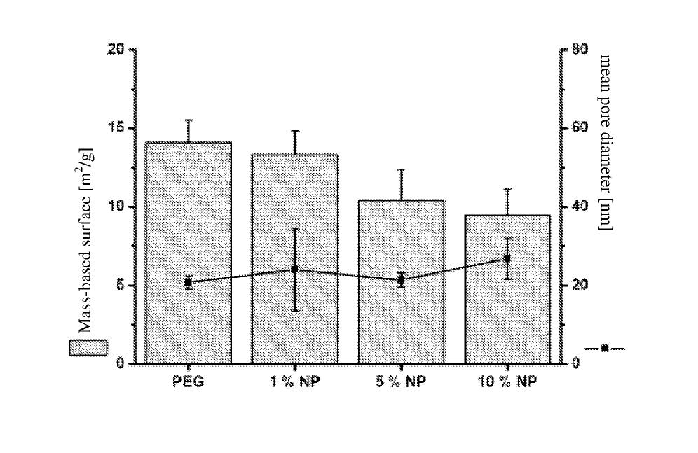

x-axis: Time (hours)

y-axis: Cumulatively released coumarin [%]

NP: Nanoparticles

PEG/NP: Nanoparticles spun into PEG fibers

PVA/NP: Nanoparticles spun into PVA fibers

PVA_{cross}/NP: Nanoparticles spun into PVA fiber, PVA cross-linked

All results with n=4; reported as the mean±SD (standard deviation).

The statistical calculations were performed with the software SigmaStat 3.5 (STATCON, Witzenhausen, Germany). To determine statistically significant differences, a one-way analysis of variance (ANOVA) with a Bonferroni post-hoc t-test analysis was performed. Probability values p<0.05 were considered statistically significant.

NP Versus PEG/NP

| Time (h) | p |
|---|---|
| 1 | 0.995 |
| 2 | 0.243 |
| 4 | 0.162 |
| 8 | 0.334 |

NP Versus PVA/NP

| Time (h) | p |
|---|---|
| 1 | 0.003 |
| 2 | 0.007 |
| 4 | <0.001 |
| 8 | 0.084 |

NP Versus PVA_{cross}/NP

| Time (h) | p |
|---|---|
| 1 | 0.029 |
| 2 | 0.001 |
| 4 | 0.001 |
| 8 | 0.338 |

In the case of PVA, both without and with cross-linking, there was a significant retardation effect over four hours.

FIG. 2

FIG. 2 shows the results of gas adsorption measurements.

x-axis: Investigated substances

y-axis (left): Mass-based surface [m²/g]

y-axis (right): Mean pore diameter [nm]

PEG: Polyethylene glycol-fiber (without nanoparticles)

1% NP: PEG fiber with 1% nanoparticles

5% NP: PEG fiber with 5% nanoparticles

10 5 NP—PEG fiber with 10% nanoparticles

### EXEMPLARY EMBODIMENTS

In Vitro Characterization: NP in Fibers

Methods—Dye

Absorption, Excitation and Emission Maximum of Coumarin 6

For the measurement of the excitation and emission fluorescence spectra, a fluorescence spectrometer LS50B from Perkin Elmer was used. The spectra of coumarin 6 solutions were recorded at room temperature at a concentration of about 30 ng/ml.

Scan range: 300-800 nm, slit 5 nm

Scan rate: about 300 nm/min

The excitation and emission wavelength were obtained from the plot of measured wavelength versus the normalized fluorescence intensity, wherein the graduation of the y-axis has been such that the maximum peak height corresponded to approximately 70% of the maximum value on this scale.

Saturation Solubility of Coumarin 6

An excess of coumarin 6 (about 20 mg) was added to 10 ml each of ethanol containing phosphate-buffered saline solution (pH 7.4) (PBS). The ethanol concentrations were 30, 50 and 100% (w/w). The suspensions were stirred for 12 h at room temperature. After equilibration, the samples were stored for 12 h in order to avoid supersaturation.

Undissolved model active ingredient (i.e. coumarin 6) was removed by centrifugation, as described below.

After appropriate dilution of each sample, the concentration of dissolved coumarin 6 was determined by fluorescence spectrometry, as described below.

Methods—NP

Production

Coumarin 6-loaded nanoparticles were produced by a modified solvent displacement method: 50 mg of PLGA were dissolved at 25° C. in 1 ml of acetone (polymer stock solution). In addition, coumarin 6 was dissolved in acetone at a concentration of 50 mg/ml (coumarin stock solution). Then, 0.5 ml of polymer stock solution (50 mg/ml) was mixed with 0.5 ml of coumarin stock solution (50 mg/ml). With stirring, the resulting solution was then injected into an aqueous phase comprising 5 ml of filtered and double-distilled water (pH 7.0, conductivity 0.055 μS/cm, 25° C.). The injection of the organic solution into the aqueous phase was carried out via an injection needle (Fine-Ject® 0.6×30 mm) using an electronically adjustable single-stage suction pump at a constant flow rate (8.0 ml/min). The pumping rate was controlled via an electronic power control and permanently monitored. After injecting the organic solution, the resulting colloidal suspension was stirred for about 3 hours at reduced pressure to remove the organic solvent. The particles were characterized immediately after production, and used.

NP Properties before Concentration

Measurement of Particle Size

The average particle size and the size distribution of the resulting nanoparticles were determined by photon correlation spectroscopy (PCS) using a NanoZS/ZEN3600 Zetasizer (Malvern Instruments).

The measurement was performed at 25° C., with samples being diluted appropriately with filtered and double-distilled water to avoid multiple scattering.

The average particle diameter (Z-Ave) and the width of the Gaussian distribution, indicated as polydispersity index (PDI), were calculated using the DTS V. 5.02 software.

Each size determination was performed at least ten times. All measurements were performed in triplicate directly after the production of the nanoparticles.

Measurement of the ζ-potential

The ζ-potential was measured by laser Doppler anemometry (LDA) with a Zetasizer NanoZS/ZEN3600 (Malvern Instruments).

The measurement was performed at 25° C. with samples appropriately diluted with 1.56 mM NaCl solution to ensure a constant ionic strength. The average values of the ζ-potential were determined from the data of multiple measurements using DTS V. 5.02 software. All measurements were performed in triplicate directly after the production of the nanoparticles.

Atomic Force Microscopy (AFM)

The morphology of the nanoparticles was determined by atomic force microscopy (AFM).

The samples were prepared by placing a sample volume of 10 μl onto a commercial slide (RMS<3 mm). The slides were incubated for 10 min with the nanoparticle suspension, then rinsed twice with distilled Water and dried in a stream of dry nitrogen. The samples were measured within 2 hours after production.

For AFM measurements, a NanoWizard® (JPK Instruments) was used in the intermittent contact mode to avoid damaging the sample surface. Commercially available Si3N4 tips attached to I-type cantilevers with a length of 230 μm and a nominal force constant of 40 N/m were used (NSC16 AIBS, Micromasch, Tallinn). The scan frequency was between 0.5 Hz and 1 Hz and was inversely proportional to scan size. The results were presented as trace signal in the amplitude mode.

NP Properties after Concentration

See Above

Concentrating the Nanoparticle Suspension

Before carrying out the electro-spinning experiments, the nanoparticle suspension was concentrated. 6 ml of nanoparticle suspension were (5 mg/ml) was placed in Vivaspin 6 ultrafiltration columns (100,000 MWCO) (Sartorius) and centrifuged for 15 minutes at 1,000×g to a final volume of 2 ml (15 mg/ml) was centrifuged. The particles were characterized immediately after production and used.

Concentration factor=NP recovery after concentration/NP recovery before concentration

AFM

See Above

NP-Recovery

Determination of Nanoparticle Recovery

The nanoparticle recovery was calculated by gravimetrical determination of the remaining nanoparticle mass after production.

Samples of 175 μl of nanoparticle suspension were ultracentrifuged at 110,000 rpm (199,000×g) for 30 minutes at ° C. After centrifugation, the supernatant was removed and the remaining pellet was freeze-dried to a constant mass (Beta II, Christ). Nanoparticle recovery (%) was calculated according to the following equation:

### Nanoparticle Recovery (%)=(mass of nanoparticles/mass of the polymer introduced into the system)×100

Active Ingredient Content and Encapsulation Efficiency

Weighed pellet was dissolved in acetonitrile, the solution is diluted with acetonitrile and measured fluorimetrically against a standard series of known concentrations of coumarin 6 in acetonitrile (LS50B, Perkin Elmer)

Methods—Fibers

Production

Nanoparticles loaded with active ingredient can be processed together with a biocompatible polymer to form nanoparticles that are loaded with active ingredient and spun into biocompatible nanofibers.

A polymer solution (about 5% (w/v) and an aqueous suspension of nanoparticles (0%, 1% and 10% by weight in water) are spun together.

Electrode distance: 20 cm; voltage: 25 kV

Cross-Linking of PVA Fibers

Next, PVA fibers were cross-linked with glutaraldehyde.

Active Ingredient Content and Encapsulation Efficiency

Known amounts of fibers are added to 0.5 ml of water. Next, 1 ml of chloroform is added. After approximately 24 h a sample is taken from the chloroform phase and analyzed fluorimetrically.

BET Surface Area and Pore Size

Gas Adsorption Measurements

Gas adsorption measurements were performed with a BELSORP-mini (BEL Japan) in the high precision mode. In this mode, saturation vapor pressure and dead volume are subtracted from the measured value. BELSORP-Mini uses a volumetric gas adsorption method. The samples were prepared by heating for 24 h at 25° C. under vacuum. Measurements of dead volumes were performed at room temperature using helium gas. Adsorption-desorption measurements were performed with the sample cell and with an empty reference cell. Both cells were immersed in liquid nitrogen to ensure a constant temperature (−196° C.). The dead volume changes because of the removal of liquid nitrogen under vacuum. Therefore, before each adsorption measurement, the dead volume of the reference cell was determined. Gaseous nitrogen was used as adsorbent.

CLSM

Confocal Laser Scanning Microscopy (CLSM)

To visualize the distribution of the nanoparticles in the nanofiber nonwovens laser scanning microscopy (CLSM) was performed. The fiber mats were fixed on a slide without fixation reagent to exclude any destruction of the fibers. A Zeiss LSM 510 scan module-coupled Zeiss Axiovert 100 M microscope was used.

An argon laser with an excitation wave of 488 nm was used for excitation of the coumarin-6 fluorescence. The transmitted light was used for visualization of the structures of the nanofiber nonwovens. A number of optical sections were obtained and analyzed using Zeiss LSM 510 ™ software (Zeiss, Jena).

Release

Transfer of a known amount of loaded (10% NP loaded with dye) fibers (about 50 mg) in 10 ml of EtOH/PBS (1+1, m/m). Incubation of the sample at 37° C. with agitation (Rotatherm, 20 rpm). After 1, 2, 4, 8, 16 and 24 hours, respectively, 175 μl of sample was removed, centrifuged (see above), diluted and analyzed fluorimetrically against a standard series of coumarin 6 in EtOH/PBS.

1. Composite materials comprising polymer fibers and polymer nanoparticles, wherein
the polymer nanoparticles and the polymer fibers comprise of biocompatible polymers,
at least one of polymer materials is loaded with at least one substance selected from therapeutic and diagnostic agents
therapeutic and diagnostic agents are selected from lipophilic and hydrophilic substances, characterized in that
the polymer nanoparticles have diameters of 10 nm to 600 nm,
the polymer fibers have diameters of 10 nm to 50 mm and lengths of 1 μm to several meters,
the polymer nanoparticles comprise a first polymer and the polymer nanofibers comprise a second polymer,
first and second polymer are selected from hydrophilic and lipophilic polymers,
first and second polymer are identical or different, and
the at least one polymer material and the at least one substance, with which it is loaded, are both hydrophilic, or are both lipophilic.

2. The composite materials according to claim 1, characterized in that the first and the second polymer are different from each other.

3. The composite materials according to claim 1, characterized in that the first and the second polymer are different from each other, wherein one of the polymers is hydrophilic and the other is hydrophobic.

4. The composite materials according to claim 1, characterized in that the first and the second polymer are biodegradable.

5. The composite materials according to claim 1, characterized in that the polymer nanoparticles are loaded with exactly one substance selected from therapeutic and diagnostic agents, while the polymer fibers are not loaded.

6. The composite materials according to claim 1, characterized in that both the polymer nanoparticles and the polymer fibers are loaded each with exactly one substance selected from therapeutic and diagnostic agents, wherein the fibers are loaded with a fast-releasing substance and the particles are loaded with a slow-releasing substance.

7. The composite materials according to claim 1, characterized in that at least the polymer nanoparticles are loaded and that both the first polymer and the at least one substance selected from diagnostic and therapeutic agents, with which the particles are loaded, are both lipophilic.

8. The composite materials according to claim 1, characterized in that the second polymer that makes up the polymer fibers, is cross-linked.

9. A process for producing composite materials according to claim 1, comprising the following steps:
a) producing nanoparticles from a first polymer, wherein the nanoparticles are optionally loaded with at least one substance selected from therapeutic and diagnostic agents,
b) mixing of the optionally loaded polymer nanoparticles of step a) with a second polymer,
c) optionally adding at least one substance selected from therapeutic and diagnostic agents, wherein at least in one of the steps a) and c) a substance selected from diagnostic and therapeutic agents is added,
d) processing the mixture of step c) into composites comprising polymer fibers and polymer nanoparticles.

10. The process according to claim 10, characterized in that the polymer nanoparticles are produced by controlled precipitation and the composites according to the invention are produced by electro-spinning.

11. A use of composite materials according to claim 1 for producing a pharmaceutical or a medicinal product.
